(19) United States
(12) Patent Application Publication
(10) Pub. No.: US 2023/0098823 A1
(43) Pub. Date: Mar. 30, 2023

(54) STEERING JOINT AND ENDOSCOPE

(71) Applicant: Medimaging Integrated Solution, Inc., Hsinchu (TW)

(72) Inventors: CHU-MING CHENG, Hsinchu (TW); CHIA-JUNG LEE, Hsinchu (TW); YUEH-YING FAN, Hsinchu (TW); YI-MING TAI, Hsinchu (TW); CHUNG-HAU WANG, Hsinchu (TW)

(21) Appl. No.: 17/940,295

(22) Filed: Sep. 8, 2022

(30) Foreign Application Priority Data
Sep. 24, 2021 (TW) 110135704

(51) Int. Cl. A61B 1/005

(52) U.S. Cl. CPC A61B 1/0055; A61B 2017/00323

(57) ABSTRACT

A steering joint comprises a first tubular member and a second tubular member. The first tubular member and the second tubular member include a plurality of cuts to facilitate bending. One end of the first tubular member has an accommodation portion for accommodating an endoscopic image module, and the other end has a first joint portion. One end of the second tubular member has a second joint portion which is joined to the first joint portion of the first tubular member. The tubular member of the steering joint is easy to injection mold and can reduce the accumulated tolerance. An endoscope including the abovementioned steering joint is also disclosed.

### BACKGROUND OF THE INVENTION

### 1. Field of the Invention

The present invention relates to a steering joint, particularly to a steering joint applicable to endoscopes.

### 2. Description of the Prior Art

An endoscope can reach cavities, which the naked eyes are unlikely to observe directly. Therefore, endoscopes have been extensively used in industry and medicine. Especially, the application of endoscopes to medicine favors medical diagnosis very much. An endoscope may access cavities of a human body through tiny channels of the human body. For an example, an endoscope may reach the lung through a bronchial tube. For another example, an endoscope may enter the bladder through the urinary tract. Hence, miniaturizing the endoscopic steering joint has become an important subject of the concerned field.

A conventional endoscopic steering joint comprises a plurality of annuluses structured identically and connected in sequence by a steering control line. For the abovementioned structure, miniaturized annuluses are hard to assemble and likely to have a larger accumulated tolerance. Thus, the conventional endoscopic steering joint makes the endoscope hard to operate smooth. Another conventional endoscopic steering joint is in form of a tubular structure, which is fabricated into a one-piece component and has a plurality of cut grooves on the tube wall. The cut grooves favor the bending of the steering joint. However, the injection molding technology is less likely to produce a long and thin steering joint meeting the requirement of miniaturization. Besides, the mold may be consumed fast, which would raise the fabrication cost.

Accordingly, an easy-to-produce miniaturized steering joint has become a target the manufacturers are eager to achieve.

### SUMMARY OF THE INVENTION

The present invention provides a steering joint, which is formed by connecting a plurality of tubular structures each having a smaller aspect ratio, whereby the steering joint can be easily produced in an injection molding technology, and whereby is simplified the assembling process of the steering joint and reduced the accumulated tolerance of the steering joint. The present invention also provides an endoscope using the same.

One embodiment of the present invention provides a steering joint, which comprises a first tubular member and a second tubular member. The first tubular member includes a first opening, a second opening, a plurality of first cuts, and a plurality of first line slots. The end of the first opening has an accommodation portion for accommodating an endoscopic image module. The plurality of first cuts is diametrically opposite to each other to define a plurality of first connection segments and a plurality of first annulus structures. The plurality of first line slots axially passes through the plurality of first annulus structures. The end of the second opening has a first joint portion, and the first line slot passes through the first joint portion. The second tubular member includes a third opening, a fourth opening, a plurality of second cuts, and a plurality of second line slots. The end of the third opening has a second joint portion, which is to be connected with the first joint portion of the first tubular member. The plurality of second cuts is diametrically opposite to each other to define a plurality of second connection segments and a plurality of second annulus structures. The plurality of second line slots axially passes through the plurality of second annulus structures and is corresponding to the first line slots. A plurality of steering control lines is disposed inside the plurality of first line slots and the plurality of second line slots for steering the first opening of the first tubular member.

Another embodiment of the present invention provides an endoscope, which comprises an endoscopic image module, a steering joint, a plurality of steering control lines, a plurality of electric cables and an electric connector. The endoscopic image module can be extended to a cavity to capture images inside the cavity and generate corresponding electronic signals. The steering joint includes a first tubular member and a second tubular member. The first tubular member includes a first opening, a second opening, a plurality of first cuts, and a plurality of first line slots. The end of the first opening has an accommodation portion for accommodating the endoscopic image module. The plurality of first cuts is diametrically opposite to each other to define a plurality of first connection segments and a plurality of first annulus structures. The plurality of first line slots axially passes through the plurality of first annulus structures. The end of the second opening has a first joint portion, and the first line slot passes through the first joint portion. The second tubular member includes a third opening, a fourth opening, a plurality of second cuts, and a plurality of second line slots. The end of the third opening has a second joint portion, which is to be connected with the first joint portion of the first tubular member. The plurality of second cuts is diametrically opposite to each other to define a plurality of second connection segments and a plurality of second annulus structures. The plurality of second line slots axially passes through the plurality of second annulus structures and is corresponding to the first line slots. The plurality of steering control lines is disposed inside the plurality of first line slots and the plurality of second line slots for steering the first opening of the first tubular member. The plurality of electric cables is electrically connected with the endoscopic image module. The electric connector is electrically connected with the plurality of electric cables. The endoscope of the present invention may be electrically connected with an external electronic device through the electric connector in a pluggable way.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 is a diagram schematically showing a steering joint according to a first embodiment of the present invention;

FIG. 2 is a diagram schematically showing an assembled steering joint according to the first embodiment of the present invention;

FIG. 3 is a side view schematically showing a steering joint according to the first embodiment of the present invention;

FIG. 4a is a locally-enlarged view schematically showing a steering joint according to the first embodiment of the present invention;

FIG. 4b is a locally-enlarged view schematically showing a steering joint according to a second embodiment of the present invention;

FIG. 4c is a locally-enlarged view schematically showing a steering joint according to a third embodiment of the present invention;

FIG. 5 is a diagram schematically showing a steering joint according to a fourth embodiment of the present invention;

FIG. 6 is a diagram schematically showing a steering joint according to a fifth embodiment of the present invention; and

FIG. 7 is a diagram schematically showing an endoscope according to one embodiment of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

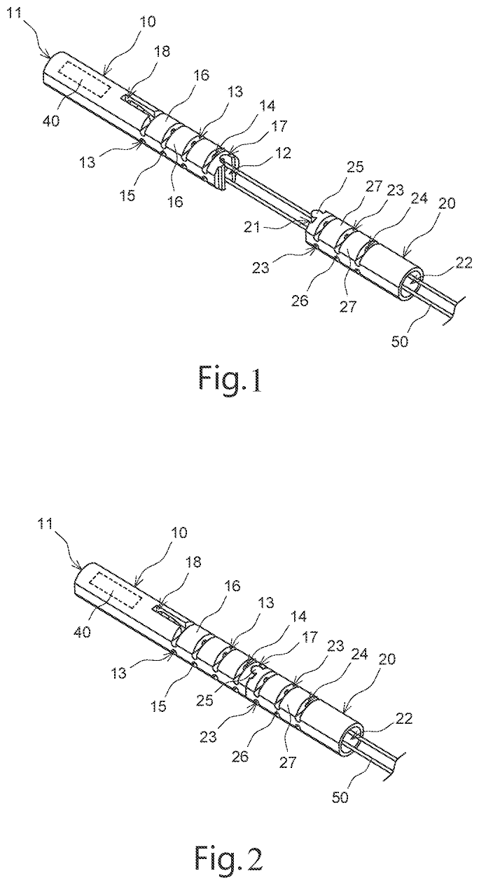

Refer to FIG. 1 and FIG. 2 for a steering joint according to a first embodiment of the present invention. The steering joint of the first embodiment is applied to an endoscope and comprises a first tubular member 10 and a second tubular member 20. The first tubular member 10 includes a first opening 11, a second opening 12, a plurality of first cuts 13, and a plurality of first line slots 14. The end of the first opening 11 has an accommodation portion for accommodating an endoscopic image module 40. The end of the second opening 12 has a first joint portion 17. The plurality of first cuts 13 is diametrically opposite to each other to define a plurality of first connection segments 15 and a plurality of first annulus structures 16. The plurality of first annulus structures 16 is connected by the plurality of first connection segments 15. The plurality of first line slots 14 axially passes through the plurality of first annulus structures 16. In one embodiment, the first cut 13 may be a U-shaped notch or a V-shaped notch. In other words, the internal surfaces of the first cut 13, which are opposite to each other, are almost parallel to each other or have an included angle opened to the exterior.

The second tubular member 20 includes a third opening 21, a fourth opening 22, a plurality of second cuts 23, and a plurality of second line slots 24. The plurality of second cuts 23 is diametrically opposite to each other to define a plurality of second connection segments 26 and a plurality of second annulus structures 27. The plurality of second annulus structures 27 is connected by the plurality of second connection segments 26. In one embodiment, the second cut 23 may be a U-shaped notch or a V-shaped notch. In other words, the internal surfaces of the second cut 23, which are opposite to each other, are almost parallel to each other or have an included angle opened outwards. The plurality of second line slots 24 axially passes through the plurality of second annulus structures 27 and is corresponding to the first line slots 14. A plurality of steering control lines 50 may be disposed inside the plurality of first line slots 14 and the plurality of second line slots 24 for steering the first opening 11 of the first tubular member 10. In one embodiment, a proximal end of the accommodation portion of the first tubular member 10 has a plurality of fixing slots 18, and the ends of the plurality of steering control lines 50 are fixed in the fixing slots 18. Based on the abovementioned structure, pulling one of the steering control lines 50 may decrease the spacing of the first cuts 13 and the spacing of the second cuts 23 on the same side to change the direction of the first opening 11 of the first tubular member 10.

The end of the third opening 21 has a second joint portion 25. The first tubular member 10 and the second tubular member 20 may be produced to be a steering joint having appropriate length via the mutual joint of the first joint portion 17 and the second joint portion 25. In the embodiment shown in FIG. 1, the first joint portion 17 is a recess, and the second joint portion 25 is a protrusion, whereby the first joint portion 17 and the second joint portion 25 may be press-fitted to each other. It is easily understood: the first joint portion 17 may be a protrusion, and the second joint portion 25 may be a recess. Thus, the first joint portion 17 and the second joint portion 25 may also be press-fitted to each other.

It is easily understood: the steering joint should have a sufficient number of the first annulus structures 16 and a sufficient number of the second annulus structures 27 so that the steering joint can achieve a required bend angle and a required bend radius. Based on the abovementioned structure, the steering joint of the present invention can acquire a sufficient number of the first annulus structures 16 and a sufficient number of the second annulus structures 27 merely via joining the first tubular member 10 and the second tubular member 20. Thus is reduced the tolerance accumulated in assemblage. Besides, a single first annulus structure 16 or a single second annulus structure 27 has a smaller aspect ratio. Therefore, the first annulus structures 16 and the second annulus structures 27 can be injection-molded easily, and the molds thereof are worn more slowly. In brief, the steering joint of the present invention decreases the fabrication cost significantly, has higher assembling precision and operates smoother.

Refer to FIG. 1 and FIG. 2 again. In one embodiment, the plurality of first line slots 14 is arranged with respect to the plurality of first connection segments 15 by an angle of 90 degrees. Based on the structure, the force pulling the steering control lines 50 may effectively make the first annulus structures 16 rotate with respect to the first connection segments 15, whereby to compress the spacing of the openings of the first cuts 13 and change the direction of the first opening 11 of the first tubular member 10. It is easily understood: as the plurality of second line slots 24 is arranged corresponding to the plurality of first line slots 14, the plurality of second line slots 24 is arranged with respect to the plurality of second connection segments 26 by an angle of 90 degrees. In one embodiment, the plurality of first line slots 14 passes through the first joint portion 17 to enhance the reliability and tightness of the connection between the first joint portion 17 and the second joint portion 25.

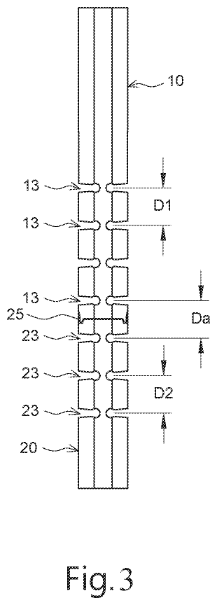

Refer to FIG. 3. In one embodiment, first spacings D1 between the axially neighboring first cuts 13 are identical. However, the present invention is not limited by the embodiment. In one embodiment, first spacings D1 between the axially neighboring first cuts 13 are different. Similarly, second spacings D2 between the axially neighboring second cuts 23 may be identical or different. In one embodiment, a spacing Da between the first cut 13 and the axially neighboring second cut 23 is identical to the first spacing D1 between the first cuts 13 or the second spacing D2 between the second cuts 23. It is easily understood: the spacing Da may be greater than the first spacing D1 or the second spacing D2 so as to enhance the structural strength between the first joint portion 17 of the first tubular member 10 and the second joint portion 25 of the second tubular member 20. In one embodiment, the first spacing D1 of the plurality of first cuts 13 is smaller than the second spacing D2 of the plurality of second cuts 23 so as to increase the bend radius of the distal end (near the first opening 11 of the first tubular member 10).

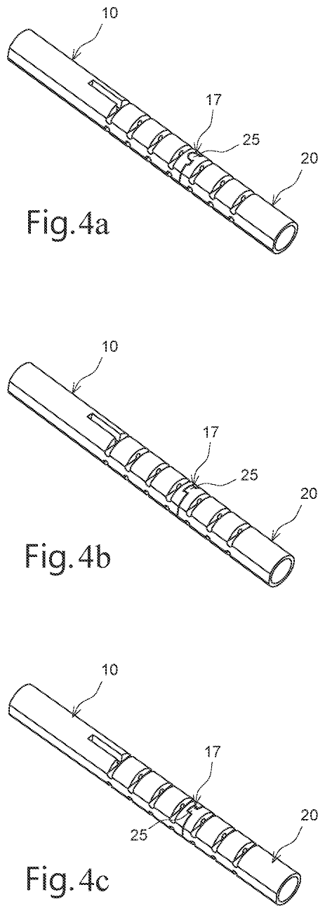

Refer to FIGS. 4a-4c for the first joint portion 17 of the first tubular member 10 and the second joint portion 25 of the second tubular member 20, which respectively have different structures, wherein the first joint portion 17 is a recess and the second joint portion 25 is a protrusion. In the embodiment shown in FIG. 4a, the second joint portion 25 is a protrusion having an arc larger than 180 degrees. Therefore, the greatest width of the second joint portion 25 is larger than the width of the connection region of the second joint portion 25. The first joint portion 17 is a recess corresponding to the second joint portion 25. After the press-fitting of the first joint portion 17 and the second joint portion 25, an interlocking is axially formed between the first joint portion 17 and the second joint portion 25. In other words, the first tubular member 10 and the second tubular member 20 are less likely to separate in the axial direction.

In the embodiment shown in FIG. 4b, the second joint portion 25 is an inverse-trapezoid protrusion. In other words, the greatest width of the second joint portion 25 is larger than the width of the connection region of the second joint portion 25. The first joint portion 17 is a recess corresponding to the second joint portion 25. After the press-fitting of the first joint portion 17 and the second joint portion 25, an interlocking is axially formed between the first joint portion 17 and the second joint portion 25. Therefore, the first tubular member 10 and the second tubular member 20 are less likely to separate in the axial direction. In the embodiment shown in FIG. 4c, the second joint portion 25 is a protrusion having a hook, and the greatest width of the second joint portion 25 is larger than the width of the connection region of the second joint portion 25. The first joint portion 17 is a recess corresponding to the second joint portion 25. After the press-fitting of the first joint portion 17 and the second joint portion 25, an interlocking is axially formed between the first joint portion 17 and the second joint portion 25. Therefore, the first tubular member 10 and the second tubular member 20 are less likely to separate in the axial direction.

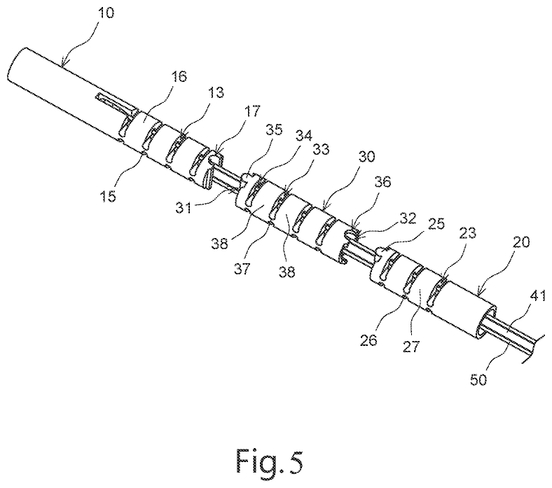

Refer to FIG. 5. In one embodiment, the steering joint of the present invention further comprises a third tubular member 30. The third tubular member 30 is disposed between the first tubular member 10 and the second tubular member 20. The third tubular member 30 includes a fifth opening 31, a sixth opening 32, a plurality of third cuts 33, and a plurality of third line slots 34. The end of the fifth opening 31 has a third joint portion 35, which is to be joined with the first joint portion 17 of the first tubular member 10. The end of the sixth opening 32 has a fourth joint portion 36, which is to be joined with the second joint portion 25 of the second tubular member 20. The plurality of third cuts 33 is diametrically opposite to each other to define a plurality of third connection segments 37 and a plurality of third annulus structures 38. The plurality of third annulus structures 38 is connected by the plurality of third connection segments 37. In one embodiment, the third cut 33 may be a U-shaped notch or a V-shaped notch. In other words, the internal surfaces of the third cut 13, which are opposite to each other, are almost parallel to each other or have an included angle opened to the exterior. The plurality of third line slots 34 axially passes through the plurality of third annulus structures 38 and is corresponding to the plurality of first line slots 14 and the plurality of second line slots 24. It is easily understood: a plurality of third tubular members may be arranged between the first tubular member 10 and the second tubular member 20 to form a steering joint having the desired length.

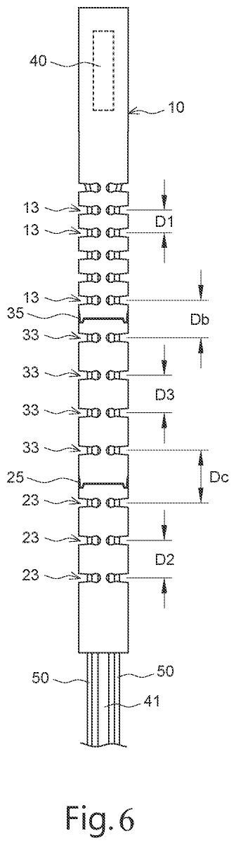

Refer to FIG. 6. In one embodiment, third spacings D3 between the axially neighboring third cuts 33 may be identical or different. In one embodiment, the first spacing D1 of the plurality of first cuts 13 is smaller than or equal to the third spacing D3 of the third cuts 33. In one embodiment, the third spacing D3 of the third cuts 33 is smaller than or equal to the second spacing D2 of the plurality of second cuts 23. In one embodiment, the first spacing D1 of the plurality of first cuts 13 is smaller than the third spacing D3 of the third cuts 33, and the third spacing D3 of the third cuts 33 is smaller than the second spacing D2 of the plurality of second cuts 23, whereby the distal end of the steering joint has a smaller bend radius, and the steering joint can keep an appropriate rigidity. In one embodiment, a spacing Db between the first cut 13 and the axially neighboring third cut 33 is larger than or identical to the first spacing D1 or the third spacing D3 so as to enhance the structural strength between the first joint portion 17 of the first tubular member 10 and the third joint portion 35 of the third tubular member 30. Similarly, a spacing Dc between the third cut 33 and the axially neighboring second cut 23 may be larger than or identical to the third spacing D3 or the second spacing D2 so as to enhance the structural strength between the fourth joint portion 36 of the third tubular member 30 and the second joint portion 25 of the second tubular member 20.

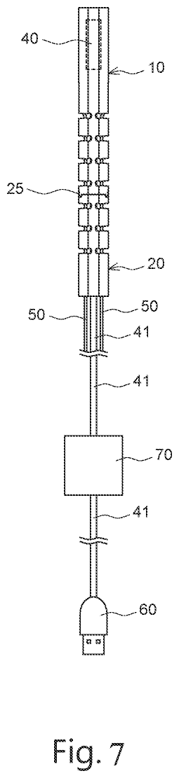

Refer to FIG. 1 and FIG. 7. In one embodiment, the endoscope of the present invention comprises an endoscopic image module 40, a steering joint, a plurality of steering control lines 50, a plurality of electric cables 41 and an electric connector 60. The structure of the steering joint has been described in detail above and will not repeat herein. The endoscopic image module 40 is disposed in the accommodation portion of the first opening 11 of the first tubular member 10. The endoscopic image module 40 can be extended to a cavity, such as a cavity of a human body or a small space in industrial inspection, to capture images inside the cavity and generate corresponding electronic signals. The endoscopic image module 40 includes an image sensor and illumination elements, which are necessary to perform the function of the endoscopic image module 40. The persons skilled in the art may use the existing technologies to realize the endoscopic image module 40. Therefore, the details of the endoscopic image module 40 will not repeat herein.

The plurality of steering control lines 50 is disposed inside the plurality of first line slots 14 of the first tubular member 10 and the plurality of second line slots 24 of the second tubular member 20 for steering the end of the first opening 11 of the first tubular member 10. The plurality of electric cables 41 is electrically connected with the endoscopic image module 40. The electric connector 60 is electrically connected with the plurality of electric cables 41. The endoscope of the present invention may be electrically connected with an external electronic device, such as a computer, a mobile Internet-access device or an electronic device dedicated to the endoscope, through the electric connector 60 in a pluggable way. Based on the abovementioned structure, the endoscope of the present invention can be cleaned and sterilized easily or used as a disposable device. In one embodiment, the electric connector 60 may be a USB interface, a connection interface of an Internet-access device, or another appropriate electric connector. In one embodiment, the endoscope of the present invention further comprises an electronic element 70, which is electrically connected with the endoscopic image module 40 and the electric connector 60. The electronic element 70 can process the electronic signals generated by the endoscopic image module 40 to facilitate the external electronic device to perform further treatment. In one embodiment, the endoscope of the present invention further comprises an operation member coupled to the plurality of steering control lines 50 to facilitate the operator to hold the and operate the endoscope. The persons killed in the art can use the existing technologies to realize the operation member. Therefore, the details of the operation member will not repeat herein.

In conclusion, the steering joint of the present invention comprises a plurality of tubular members having smaller aspect ratios, which favors injection molding and decelerates mold wearing. Besides, fewer tubular members can simplify the assembling process, decrease accumulated tolerance, and make operation smoother.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. A steering joint, comprising
a first tubular member, including a first opening, a second opening, a plurality of first cuts, and a plurality of first line slots, wherein an end of the first opening has an accommodation portion for accommodating an endoscopic image module; the plurality of first cuts is diametrically opposite to each other to define a plurality of first connection segments and a plurality of first annulus structures; the plurality of first line slots axially passes through the plurality of first annulus structures; an end of the second opening has a first joint portion, and the first line slot passes through the first joint portion; and
a second tubular member, including a third opening, a fourth opening, a plurality of second cuts, and a plurality of second line slots, wherein an end of the third opening has a second joint portion, which is to be connected with the first joint portion of the first tubular member; the plurality of second cuts is diametrically opposite to each other to define a plurality of second connection segments and a plurality of second annulus structures; the plurality of second line slots axially passes through the plurality of second annulus structures and is corresponding to the first line slots; a plurality of steering control lines is disposed inside the plurality of first line slots and the plurality of second line slots for steering the end of the first opening of the first tubular member.

2. The steering joint according to claim 1, wherein the plurality of first line slots is disposed with respect to the plurality of first connection segments by an angle of 90 degrees.

3. The steering joint according to claim 1, wherein first spacings between the plurality of axially neighboring first cuts are identical.

4. The steering joint according to claim 1, wherein second spacings between the axially neighboring second cuts are identical.

5. The steering joint according to claim 1, wherein a spacing between the first cut and the axially neighboring second cut is identical to or larger than a first spacing between the plurality of axially neighboring first cuts or a second spacing between the plurality of axially neighboring second cuts.

6. The steering joint according to claim 1, wherein a first spacing between the plurality of axially neighboring first cuts is identical to or smaller than a second spacing between the plurality of axially neighboring second cuts.

7. The steering joint according to claim 1, wherein the first cut or the second cut is a U-shaped notch or a V-shaped notch.

8. The steering joint according to claim 1, wherein a proximal end of the accommodation portion has a plurality of fixing slots for fixing ends of the plurality of steering control lines.

9. The steering joint according to claim 1, wherein the first joint portion and the second joint portion are press-fitted to each other via a protrusion and a recess.

10. The steering joint according to claim 9, wherein a greatest width of the protrusion is larger than a width of a connection region of the protrusion.

11. The steering joint according to claim 1, further comprising
at least one third tubular member, which includes a fifth opening, a sixth opening, a plurality of third cuts and a plurality of third line slots, wherein an end of the fifth opening has a third joint portion, which is to be joined with the first joint portion of the first tubular member; an end of the sixth opening has a fourth joint portion, which is to be joined with the second joint portion of the second tubular member; the plurality of third cuts is diametrically opposite to each other to define a plurality of third connection segments and a plurality of third annulus structure; the plurality of third line slots axially passes through the plurality of third annulus structures and is corresponding to the plurality of first line slots and the plurality of second line slots.

12. The steering joint according to claim 11, wherein a first spacing of the plurality of axially neighboring first cuts is smaller than or equal to a third spacing of the plurality of axially neighboring third cuts.

13. The steering joint according to claim 11, wherein a third spacing of the plurality of axially neighboring third cuts is smaller than or equal to a second spacing of the plurality of axially neighboring second cuts.

14. An endoscope, comprising
an endoscopic image module, used to extend to a cavity for capturing images inside the cavity and generating corresponding electronic signals;
a steering joint, further comprising
a first tubular member, including a first opening, a second opening, a plurality of first cuts, and a plurality of first line slots, wherein an end of the first opening has an accommodation portion for accommodating the endoscopic image module; the plurality of first cuts is diametrically opposite to each other to define a plurality of first connection segments and a plurality of first annulus structures; the plurality of first line slots axially passes through the plurality of first annulus structures; an end of the second opening has a first joint portion, and the first line slot passes through the first joint portion; and
a second tubular member, including a third opening, a fourth opening, a plurality of second cuts, and a plurality of second line slots, wherein an end of the third opening has a second joint portion, which is to be connected with the first joint portion of the first tubular member; the plurality of second cuts is diametrically opposite to each other to define a plurality of second connection segments and a plurality of second annulus structures; the plurality of second line slots axially passes through the plurality of second annulus structures and is corresponding to the first line slots;
a plurality of steering control lines, disposed inside the plurality of first line slots and the plurality of second line slots for steering the end of the first opening of the first tubular member;
a plurality of electric cables, electrically connected with the endoscopic image module; and
an electric connector, electrically connected with the plurality of electric cables to enable the endoscope to be electrically connected with an external electronic device in a pluggable way.

15. The endoscope according to claim 14, further comprising
an electronic element, electrically connected with the endoscopic image module and the electric connector to process the electronic signals generated by the endoscopic image module.

16. The endoscope according to claim 14, wherein the plurality of first line slots is disposed with respect to the plurality of first connection segments by an angle of 90 degrees.

17. The endoscope according to claim 14, wherein first spacings between the plurality of axially neighboring first cuts are identical.

18. The endoscope according to claim 14, wherein second spacings between the axially neighboring second cuts are identical.

19. The endoscope according to claim 14, wherein a spacing between the first cut and the axially neighboring second cut is identical to or larger than a first spacing between the plurality of axially neighboring first cuts or a second spacing between the plurality of axially neighboring second cuts.

20. The endoscope according to claim 14, wherein a first spacing between the plurality of axially neighboring first cuts is identical to or smaller than a second spacing between the plurality of axially neighboring second cuts.

21. The endoscope according to claim 14, wherein the first cut or the second cut is a U-shaped notch or a V-shaped notch.

22. The endoscope according to claim 14, wherein a proximal end of the accommodation portion has a plurality of fixing slots for fixing ends of the plurality of steering control lines.

23. The endoscope according to claim 14, wherein the first joint portion and the second joint portion are press-fitted to each other via a protrusion and a recess.

24. The endoscope according to claim 23, wherein a greatest width of the protrusion is larger than a width of a connection region of the protrusion.

25. The endoscope according to claim 14, wherein the steering joint further comprises
at least one third tubular member, which includes a fifth opening, a sixth opening, a plurality of third cuts and a plurality of third line slots, wherein an end of the fifth opening has a third joint portion, which is to be joined with the first joint portion of the first tubular member; an end of the sixth opening has a fourth joint portion, which is to be joined with the second joint portion of the second tubular member; the plurality of third cuts is diametrically opposite to each other to define a plurality of third connection segments and a plurality of third annulus structure; the plurality of third line slots axially passes through the plurality of third annulus structures and is corresponding to the plurality of first line slots and the plurality of second line slots.

26. The endoscope according to claim 25, wherein a first spacing of the plurality of axially neighboring first cuts is smaller than or equal to a third spacing of the plurality of axially neighboring third cuts.

27. The endoscope according to claim 25, wherein a third spacing of the plurality of axially neighboring third cuts is smaller than or equal to a second spacing of the plurality of axially neighboring second cuts.
